(19) United States
(12) Patent Application Publication
(10) Pub. No.: US 2025/0041083 A1
(43) Pub. Date: Feb. 6, 2025

(54) MEDICAL STENT WITH POST INTERVENTION INCREASING DIAMETER

(71) Applicant: KONINKLIJKE PHILIPS N.V., EINDHOVEN (NL)

(72) Inventor: Rene Leonardus Jacobus Marie UBACHS, EINDHOVEN (NL)

(21) Appl. No.: 18/718,082

(22) PCT Filed: Dec. 7, 2022
(86) PCT No.: PCT/EP2022/084688

§ 371 Date: Jun. 10, 2024

Related U.S. Application Data
(60) Provisional application No. 63/290,372, filed on Dec. 16, 2021.

(51) Int. Cl. A61F 2/852

(52) U.S. Cl. CPC A61F 2/852; A61F 2210/0004; A61F 2210/0014; A61F 2210/0061

(57) ABSTRACT

A stent device (10) includes a main stent body (12) configured to expand when placed in a treatment site of a patient, the main stent body configured to expand to a first diameter (D1) at a first time and a second diameter (D0) at a later second time. The second diameter is greater than the first diameter.

### FIELD

The following relates generally to the vascular stent arts, vascular stent delivery arts, and related arts.

### BACKGROUND

Arteries and veins can develop stenosis, i.e. the lumen becomes obstructed and blood flow is consequently blocked. A common therapy is to increase the lumen through balloon angioplasty, followed by the implantation of a stent to ensure the created patency is maintained. The diameter of the selected stent should be large enough to ensure good contact with the vessel walls with enough pressure on the wall to maintain patency and ensure no migration occurs. On the other hand, the stent diameter should not be too large such that it generates too much pressure on the tissue and causes either damage to the tissue or disturbance of the blood flow through a sudden increase in lumen diameter.

After stent implantation there will generally also be some measure of in-stent restenosis, developing over the first number of months, which needs to be accounted for when selecting the diameter of the stent. In-stent restenosis can be the result of damage to the tissue and disturbance of blood flow.

The following discloses certain improvements to overcome these problems and others.

### SUMMARY

In some embodiments disclosed herein, a stent device includes a main stent body configured to expand when placed in a treatment site of a patient, the main stent body configured to expand to a first diameter (D1) at a first time and a second diameter (D0) at a later second time. The second diameter is greater than the first diameter.

In some embodiments disclosed herein, a stent device includes an expandable stent configured to expand to a second diameter; and a bioabsorbable constraint structure secured with the expandable stent and constraining the expandable stent to a first diameter that is less than the second diameter. The bioabsorbable constraint structure is made of a bioabsorbable material.

In some embodiments disclosed herein, a stent device includes an outer stent comprising a bioabsorbable material configured to expand to a first diameter D1; and an inner stent disposed within the outer stent and configured to expand to a second diameter D0. The second diameter D0 is greater than the first diameter D1.

One advantage resides in providing a self-expanding stent device whose diameter expands in two steps separated by a designed time interval that may be on the order of hours, days, or longer.

One advantage resides in providing a self-expanding stent device whose diameter expands gradually over a designed time interval that may be on the order of hours, days, or longer.

Another advantage resides in providing a stent device with multiple stent layers or materials that have different absorption rates in tissue of a patient.

Another advantage resides in providing a stent device that gradually exerts an increasing radial force against tissue of a patient.

Another advantage resides in providing a stent device that automatically increases a nominal diameter thereof over time.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a stent device in accordance with the present disclosure, including:
(A) a perspective view of a self-expanding main stent body having an expanded diameter D0;
(B) a perspective view of a bioabsorbable self-expanding stent having an expanded diameter D1 which is less than D0;
(C) a perspective view of the self-expanding stent device comprising the self-expanding main stent body of part (A) disposed coaxially inside the bioabsorbable self-expanding stent of part (B); and
(D) an end view of the self-expanding stent device of part (C).

FIG. 2 diagrammatically illustrates a graph of data of the stent device of FIG. 1.

FIGS. 3-7 diagrammatically illustrate other embodiments of the stent device of FIG. 1.

FIG. 8 shows an example flowchart of operations performed in a medical procedure using the stent device of FIG. 1.

### DETAILED DESCRIPTION

When a vascular stent is deployed, it is set into place either by self-expanding in the case of a self-expanding stent, or by action of an interior balloon that is inflated to expand the stent. In either case, the stent deployment places significant stress on the treatment site, which is typically already weakened due to plaque buildup and potentially due to prior therapy operations such as balloon angioplasty. Hence, while there is a desire to secure the stent with a high degree of force, this must be balanced by concern about overstressing the site. The stent diameter and stiffness are the two design parameters that most strongly control the deployment force.

The following discloses an improvement in which the stent is designed to expand to an initial diameter D1 at the time of deployment, and some time thereafter to expand again to a larger diameter D0>D1. In this way, the stress imposed on the treatment site is delivered in two stages. In some embodiments in which the expansion from D1 to D0 takes place gradually, the additional stress is imposed gradually.

In some embodiments disclosed herein, this two-stage stent expansion is implemented by the inclusion of bioabsorbable structural features into the stent. The bioabsorbable structure(s) initially restrict the expanded stent to the initial D1 diameter. For example, in one embodiment the stent comprises an inner Nitinol self-expanding stent designed to expand to diameter D0, which is disposed coaxially within an outer bioabsorbable stent designed to expand to smaller diameter D1. The initial deployment of this two-part stent expands to the smaller diameter D1. As the outer stent is absorbed by the patient's body the inner Nitinol stent is released and expands to the larger final diameter D0. The target time interval between expansion from D1 to D0 can be designed by designing the composition and geometry of the outer bioabsorbable stent to be absorbed over the target time interval.

In other embodiments, the bioabsorbable expansion limiting structure(s) may include bioabsorbable strut braces, encircling loops, or so forth that initially constrain the stent to expanding to the smaller D1 diameter, and which then are absorbed over a designed time interval thereby subsequently allowing the stent to expand to its larger final diameter D0.

In some variant embodiments, the main stent having natural expanded diameter D0 is also bioabsorbable, but at a slower rate than the bioabsorbable structure that initial restricts the expansion to diameter D1. In this case, after the treatment site has healed the main stent can also be eventually absorbed.

In any of the foregoing embodiments, the designed time interval for expansion from the initial smaller diameter D1 to the final larger diameter D0 can be designed using suitable experiments on calibration stents with different bioabsorbable structure(s) that differ in the choice of bioabsorbable material and/or the geometry of the bioabsorbable structures (e.g., bioabsorbable structures with different wire thicknesses, for example). By way of nonlimiting illustration, the calibration stents can be placed into an environment such as being deployed inside segments of pig vasculature of the appropriate inner lumen diameter disposed in a saline-based solution mimicking the salinity, pH, and other characteristics of human blood and maintained at human body temperature (e.g., around 36.5-37.0° C.) over the experimental time interval. For a more realistic experimental environment, the saline-based solution may include white and red blood cells, or may even be real blood (porcine, human, or the like), and/or may be flowed through the pig vasculature segment at a flow rate comparable to the expected blood flow rate in the target treatment site in human vasculature. The calibration stents can then be observed over time to determine the time interval for expansion from initial diameter D1 to final diameter D0 for each calibration stent. Such experimental tests can be used to optimize the time interval for the bioabsorbable structure(s) to degrade to a sufficient extent to release the constraint and allow the stent to expand from initial diameter D1 to final diameter D0.

In some examples, a rate of absorption of the bioabsorbable structure(s) can be performed to characterize the rate of absorption under various conditions as a material parameter. This data can then be used in combination with a computational physics model of the stent device and its intended environment (i.e., the tissue) to calculate the absorption time of the specific stent device. When using a parametric model, the design can then be manually or automatically adapted to reach the desired behavior.

With reference to FIG. 1, an illustrative self-expanding stent device 10 is shown in parts (C) and (D) of FIG. 1. The self-expanding stent device 10 comprises a non-bioabsorbable self-expanding stent 12, also referred to herein as a main stent body 12, which is shown in isolation in part (A). The main stent body 12 is disposed coaxially inside a bioabsorbable self-expanding stent 14, which is shown in isolation in part (B). The main stent body 12 may, for example, be a Nitinol stent. As seen in part (A), the main stent body stent 12 has an expanded diameter D0. By contrast, the bioabsorbable self-expanding stent 14 has a smaller expanded diameter D1. The self-expanding stent device 10 is constructed by inserting the main stent body stent 16 coaxially inside the bioabsorbable self-expanding stent 14. This coaxial arrangement is best seen in the end view of the self-expanding stent device 10 shown in part (D). Note that in part (D), the outer bioabsorbable stent 14 is shown using a dashed line while the inner main stent body 12 is shown using a solid line. The resulting self-expanding stent device 10 has an expanded diameter D1 due to the outer bioabsorbable self-expanding stent device 14 preventing the inner main stent body 12 from self-expanding to its full diameter D0. The bioabsorbable material making up the bioabsorbable outer stent 14 may be, for example, a material such as a polyester, poly-L-lactide (PLLA), polyglycolide (PGA), magnesium alloys, and tyrosine polycarbonate. In some examples, the stent 10 can be a drug-eluting stent. In some examples, the inner stent 16 of part (A) can be crimped and then placed inside of the outer stent 14 of part (B) to form the self-expanding stent device 10 of parts (C) and (D).

In one example, the inner stent 12 (i.e., the main stent body 12) can comprise a non-bioabsorbable material (such as Nitinol, Nickel-Titanium (Ni—Ti) alloys, Cobalt—Chromium—Nickel (Co—Cr—Ni) alloys, steel, and so forth), and the outer bioabsorbable stent 14 can comprise a bioabsorbable material, such as a polyester, poly-L-lactide (PLLA), polyglycolide (PGA), magnesium alloys, and tyrosine polycarbonate. The outer stent 14 is configured to be absorbed by tissue of the patient to allow the inner stent 12 to expand to the second diameter D0. For example, the bioabsorbable material of the outer stent 14 is configured to be absorbed from the first time to the second time. In another example, the bioabsorbable material of the outer stent 14 has a first bio-absorption time in a human blood vessel, the inner stent 12 comprises a second bioabsorbable material different from the bioabsorbable material of the bioabsorbable constraint structure, and the second bioabsorbable material has a longer bio-absorption time in a human blood vessel than the bioabsorbable material of the bioabsorbable constraint structure.

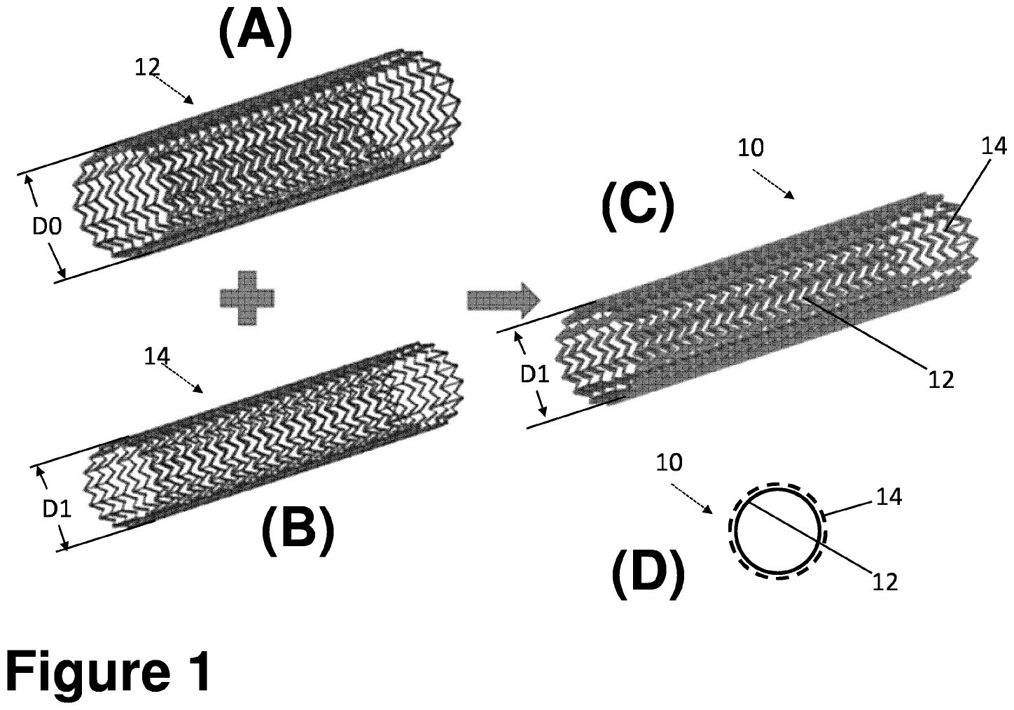

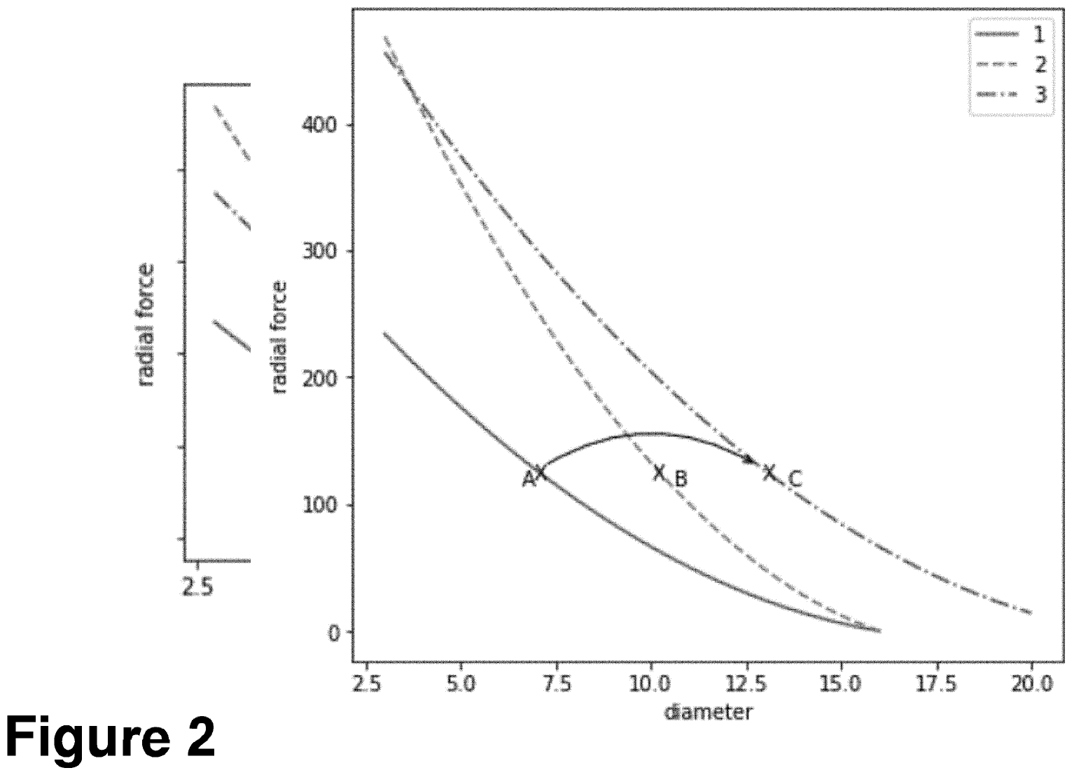

FIG. 2 shows a graph showing a radial force exerted by the main stent body 12 on tissue of a patient relative to a diameter of the main stent body 12. The stent 10 automatically increases its nominal diameter over time. As shown in FIG. 2, the stent 10 can transition from curve 1 to curve 2 to curve 3. Curve 1 shows a baseline radial force versus diameter curve for a stent. If the stiffness of this stent is increased this will result in curve 2. Here the stress-free diameter is unchanged but higher forces are exerted for all diameters below this. This would cause the vessel to be dilated more if an oversized stent is used. If less oversizing is applied, there will be less difference between lumen increase. Curve 3 shows the behavior of a stent proposed here, after the restricting material has been absorbed: similar stiffness (slope) compared to the baseline stent, but larger stress-free diameter. For a vessel with compliance corresponding to a radial force indicated by points A, B and C, placing the stent of curve 2 would result in an immediate increase of diameter to point B. Placing the proposed stent would lead to the vessel attaining diameter of point A immediately after the procedure. Over time the curve would shift towards curve 3 and correspondingly the vessel and stent diameter would increase.

In this way the amount of radial force can be increased gradually over time such that a lumen size of the main stent body 12 can also increase over time. This gradual increase allows the tissue to remodel and adapt to under moderately raised mechanical force, after which the force is increased, and the remodeled tissue once more has time to adapt to a new force. By allowing the tissue to slowly adapt, lower values of stress are imposed, and less tissue damage will be introduced.

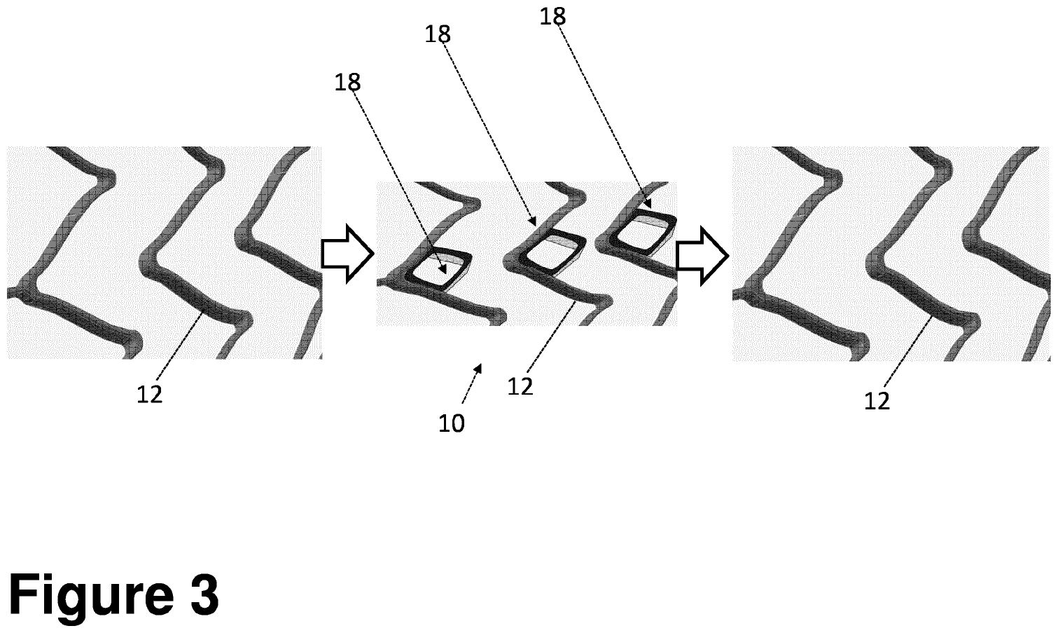

FIGS. 3-7 show different embodiments of the stent device 10. In these embodiments, the main stent body 12 is augmented by bioabsorbable structures that constrain the initial diameter of the stent device 10 to the initial diameter D1. When the bioabsorbable structures are absorbed into the bloodstream, they cease to provide stent diameter constraint, and the main stent body 12 then extends to its larger second diameter D0. FIG. 3 shows that the main stent body 12 includes bioabsorbable structures comprising one or more bioabsorbable strut braces 18. The left-hand drawing of FIG. 3 depicts a few struts of the main stent body 12 having an expanded diameter D0. The middle drawing of FIG. 3 depicts those struts constrained to a smaller diameter D1 by the bioabsorbable strut braces 18. To attach the strut braces 18, the main stent body 12 may be crimped to the smaller diameter D1 before the attachment. When the bioabsorbable strut braces 18 biodegrade, this constraint on the struts is removed and the main strut body 12 expands to its larger diameter D0, as shown in the right drawing of FIG. 3. The struts 18 can be made from the bioabsorbable material, and are added to restrict movement between the outer stent 14 and the inner stent 16. Over time, the struts 18 can be absorbed by tissue, and the inner stent 16 will expand to the second diameter D0.

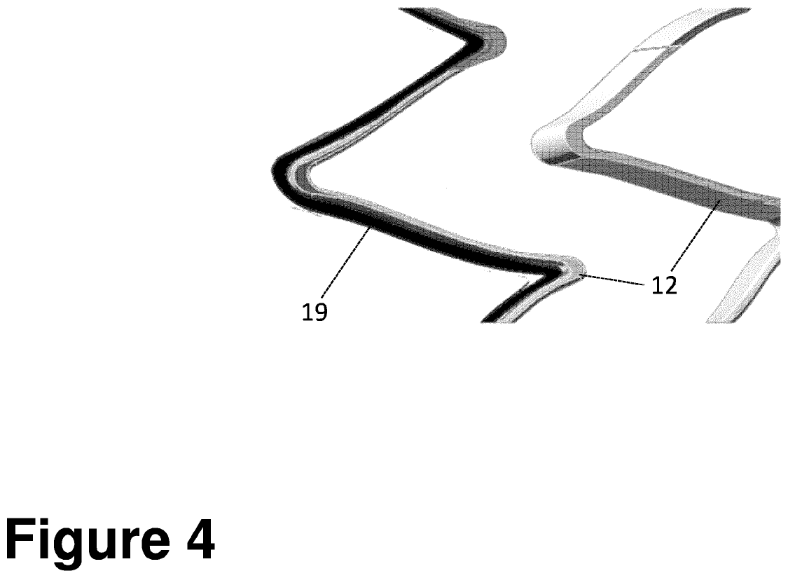

With reference to FIG. 4, in another embodiment, the struts of the main stent body 12 includes bioabsorbable structures comprising an additional layer of bioabsorbable material 19 that is added to the struts 18. The main stent body 12 may be crimped to the smaller diameter D1 before the application of the bioabsorbable material coating 19. As the coating 19 biodegrades, the constraint it imposes is removed and the main stent body 12 expands to its larger diameter D0.

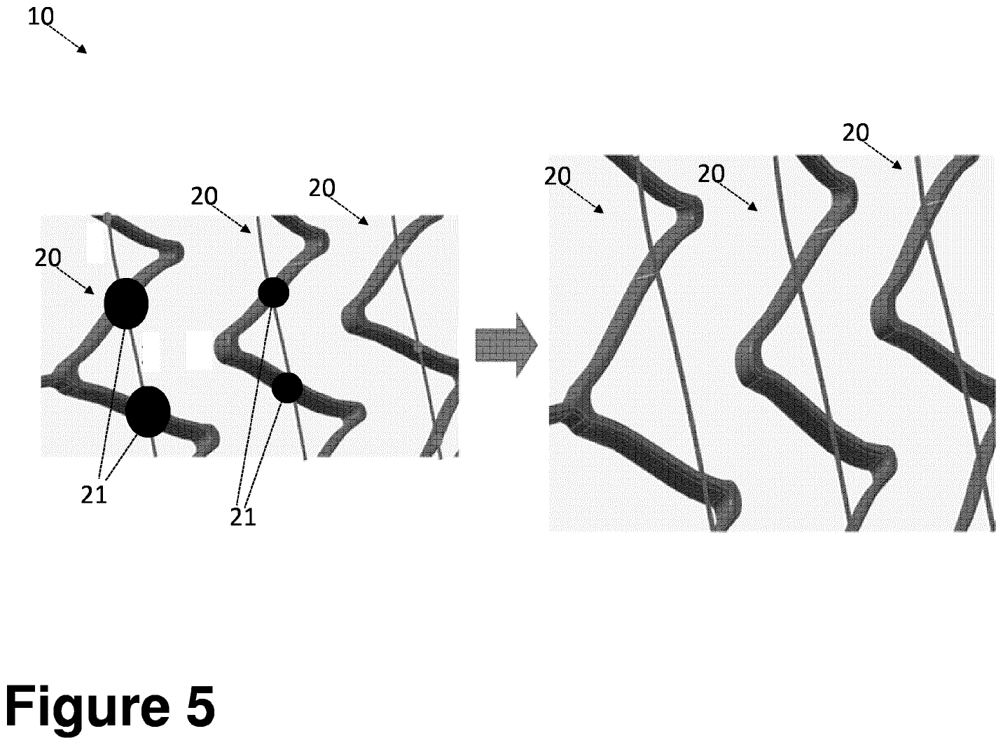

FIG. 5 shows another embodiment in which the main stent body 12 includes one or more wires formed as loops 20. The loops 20 can be made from Nitinol, and can encircle the main stent body 12. The loops 20 are connected to the main stent body 12 via the bioabsorbable material 21 at multiple positions, as seen in the left-hand drawing of FIG. 5. In some embodiments, the bioabsorbable material 21 at the different positions have different volumes of material. Consequently, they bio-absorb away at different times (i.e., the smaller masses of bioabsorbable material are absorbed first), creating a temporal sequence of “releases” of the tension provided by the loops 20, thus creating a multiple step radial force of the stent device 10 that increases over time. FIG. 5, righthand drawing, shows the illustrated struts of the expanded main stent body 12 after the bioabsorbable material has been completely absorbed.

In another embodiment, the loops 20 are themselves made of a bioabsorbable material (in which case the connections 21 may be a non-bioabsorbable material). In this case, the loops 20 themselves are absorbed into the bloodstream to enable the stent device 10 to expand from the initial diameter D1 constrained by the loops 20 to the final diameter D0 when the loops are absorbed.

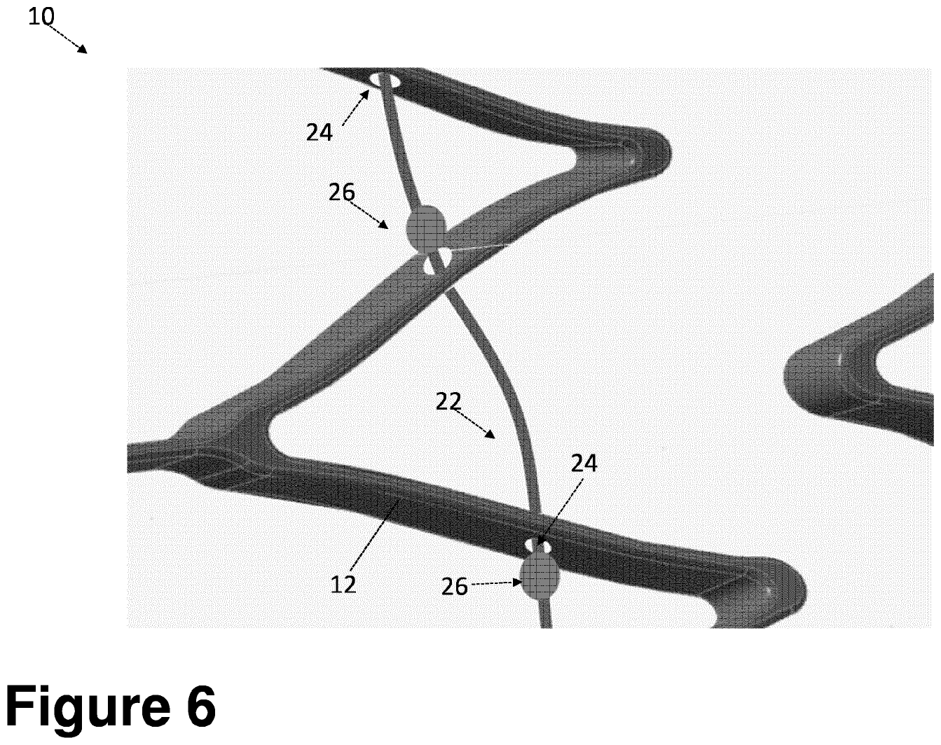

FIG. 6 shows an embodiment in which struts of the main stent body 12 include one or more bioabsorbable springs 22 made from a wire secured to the main stent body 12. A wire (one of which is shown in FIG. 6) is fixated to the stent at the distal side of the main stent body 12, lead through holes 24 (two of which are shown in FIG. 6) in main stent body 12 towards a proximal side where it is also fixated using bioabsorbable material 26. The wire(s) are run through loops in the main stent body 12 in a tangential direction. The constricted diameter of the main stent body 12 can thus be determined by the length of the wire of the spring 22. By releasing one or both of the fixated ends, the main stent body 12 will be able to expand further. In some examples, bioabsorbable stops or plugs 26 can be included to restrain the wire of the spring 22 to restrain the spring 22 from moving through one of the holes 24.

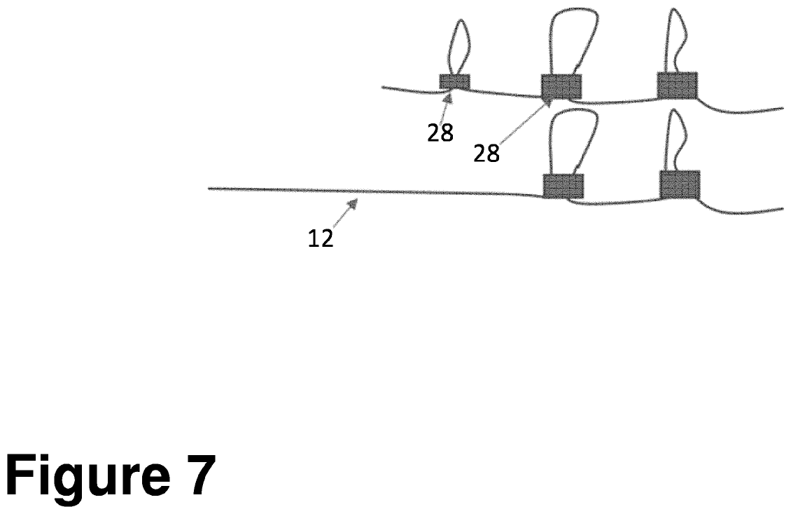

With reference to FIG. 7, the wire making up the main stent body 12 can be shortened by looping onto itself and fixating with absorbable material 28. This is done on multiple positions, again optionally with different volumes of material at different fixation points 28, thus creating a multiple step radial force increase of the stent device over time.

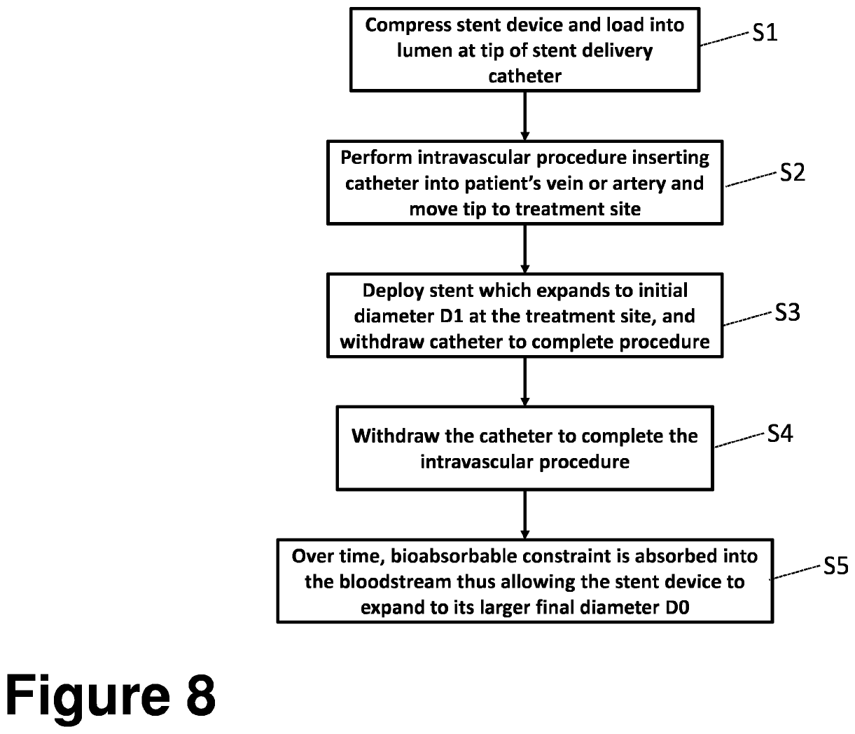

With reference to FIG. 8, a stenting procedure suitably performed with the stent device 10 is described. In an operation S1, the stent device is compressed and loaded into a lumen having diameter D2 at the tip of a stent delivery catheter. In this operation S1, the stent device 10 is compressed to the lumen diameter D2, which is less than the initial expanded diameter D1 of the expanded stent device 10.

In an operation S2, an intravascular procedure is used in which the tip of the stent delivery catheter is inserted into a vein or artery and is pushed through that vein or artery until the tip of the catheter reaches the treatment site where the stent device 10 is to be deployed. This insertion may optionally be done under guidance of medical imaging, such as computed tomography (CT) imaging or ultrasound imaging.

In an operation S3, the stent is deployed at the treatment site using a deployment control wire or other mechanism of the stent delivery catheter, which pushes the compressed stent out of the lumen of diameter D2. Upon exiting the lumen of diameter D2, the stent device 10 self-expands to its initial diameter D1, which is the diameter of the stent device 10 while constrained by the biodegradable constraint structure. In another example, the stent can also be expanded by inflating a balloon inside its lumen to the desired diameter.

In an operation S4, the delivery catheter is withdrawn to complete the intravascular procedure.

In an operation S5, the bioabsorbable constraint is absorbed into the bloodstream over time to allow the stent device to expand to its final larger diameter D0.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A stent device, comprising:
a main stent body configured to expand when placed in a treatment site of a patient, the main stent body configured to expand to a first diameter (D1) at a first time and a second diameter (D0) at a later second time, wherein the second diameter is greater than the first diameter and wherein the main stent body is constrained to the first diameter by a bioabsorbable material.

2. The stent device of claim 1, wherein the main stent body comprises Nitinol.

3. The stent device of claim 1, wherein the main stent body comprises an inner stent and further includes:
an outer stent configured to expand to the first diameter;
wherein the inner stent is disposed within the outer stent and configured to expand to the second diameter (D0).

4. The stent device of claim 3, wherein:
the inner stent comprises a non-bioabsorbable material; and
the outer stent comprises the bioabsorbable material.

5. The stent device of claim 4, wherein the outer stent is configured to be absorbed by tissue of the patient to allow the inner stent to expand to the second diameter.

6. The stent device of claim 4, wherein:
the bioabsorbable material of the outer stent has a first bio-absorption time in a human blood vessel;
the inner stent comprises a second bioabsorbable material different from the bioabsorbable material of the bioabsorbable constraint structure; and
the second bioabsorbable material has a longer bio-absorption time in a human blood vessel than the bioabsorbable material of the bioabsorbable constraint structure.

7. The stent device of claim 5, wherein the bioabsorbable material of the outer stent is configured to be absorbed from the first time to the second time.

8. The stent device of claim 1, wherein the main stent body comprises one of a strut brace or a loop, the strut brace or loop being comprises of Nitinol.

9. The stent device of claim 1, wherein the main stent body comprises the bioabsorbable material configured to constrain the main stent body to the first diameter (D1) and the main stent body expands to the second diameter (D0) upon absorption of the bioabsorbable material.

10. The stent device of claim 8, wherein the bioabsorbable material comprises a polyester, poly-L-lactide (PLLA), polyglycolide (PGA), magnesium alloys, and tyrosine polycarbonate.

11. A stent device, comprising:
an expandable stent configured to expand to a second diameter; and
a bioabsorbable constraint structure secured with the expandable stent and constraining the expandable stent to a first diameter that is less than the second diameter, the bioabsorbable constraint structure made of a bioabsorbable material.

12. The stent device of claim 11, wherein the bioabsorbable constraint structure comprises an outer stent that contains the expandable stent, the outer stent made of a bioabsorbable material and configured to expand to the first diameter.

13. The stent device of claim 11, wherein the bioabsorbable constraint structure comprises bioabsorbable springs secured to the expandable stent.

14. The stent device of claim 11, wherein the bioabsorbable constraint structure comprises bioabsorbable struts of the expandable stent.

15. The stent device of claim 11, wherein the bioabsorbable constraint structure comprises a plurality of bioabsorbable loops having the first diameter which encircle the expandable stent.

16. The stent device of claim 11, wherein the bioabsorbable material comprises a polyester, poly-L-lactide (PLLA), polyglycolide (PGA), magnesium alloys, and tyrosine polycarbonate.

17. The stent device of claim 11, wherein the expandable stent comprises a self-expanding Nitinol stent.

18. The stent device of claim 11, wherein:
the bioabsorbable material of the bioabsorbable constraint structure has a first bio-absorption time in a human blood vessel;
the expandable stent comprises a second bioabsorbable material different from the bioabsorbable material of the bioabsorbable constraint structure; and
the second bioabsorbable material has a longer bio-absorption time in a human blood vessel than the bioabsorbable material of the bioabsorbable constraint structure.

19. A stent device, comprising:
an outer stent comprising a bioabsorbable material configured to expand to a first diameter D1; and
an inner stent disposed within the outer stent and configured to expand to a second diameter D0;
wherein the second diameter D0 is greater than the first diameter D1.

20. The stent device of claim 19, wherein:
the inner stent comprises a non-bioabsorbable material.
